(19) United States
(12) Patent Application Publication
(10) Pub. No.: US 2017/0095155 A1
(43) Pub. Date: Apr. 6, 2017

(54) OBJECT INFORMATION ACQUIRING APPARATUS AND CONTROL METHOD THEREOF

(71) Applicant: CANON KABUSHIKI KAISHA, Tokyo (JP)

(72) Inventors: Takao Nakajima, Yokohama-shi (JP); Yasufumi Asao, Atsugi-shi (JP); Kohtaro Umezawa, Tokyo (JP); Toru Imai, St. Louis, MO (US)

(21) Appl. No.: 15/277,059

(22) Filed: Sep. 27, 2016

(30) Foreign Application Priority Data
Oct. 6, 2015 (JP) 2015-198218

(51) Int. Cl. A61B 5/00; A61B 5/1455; G01S 15/89

(52) U.S. Cl. CPC A61B 5/0035; G01S 15/8965; A61B 5/0095; A61B 5/7214; A61B 5/742; A61B 5/14552; A61B 2576/00

(57) ABSTRACT

An object information acquiring apparatus of the present invention includes a light source generating light having a first and a second wavelength, a conversion element receiving photoacoustic waves from an object and outputs first and second reception signals, a characteristic information acquiring unit acquiring first and second characteristic information distributions and acquiring a substance concentration distribution inside the object, a position shift acquiring unit acquiring a position shift between the first and second characteristic information distributions, and a display controlling unit displaying an image based on the substance concentration distribution and the position shift.

### BACKGROUND OF THE INVENTION

Field of the Invention

The present invention relates to an object information acquiring apparatus and a control method thereof.

Description of the Related Art

As one of imaging techniques that use light, photoacoustic imaging (PAI) is available. In the photoacoustic imaging, first, pulsed light generated from a light source is applied to an object. When the irradiation light propagates and diffuses in the object and a light absorber in the object then absorbs the energy of the light, an acoustic wave (hereinafter referred to as a photoacoustic wave) is generated. By receiving the photoacoustic wave with an ultrasound probe (transducer) and analyzing a reception signal in a processing unit, information related to an optical characteristic value of the inside of the object is acquired as image data. With this, an optical characteristic value distribution in the object is visualized.

In recent years, with the purpose of obtaining information on a minute light absorber, improvement in the resolution of the photoacoustic imaging has been demanded. To cope with this, the development of a photoacoustic microscope that images an absorber, such as a microvessel, in the vicinity of the surface of the object with high resolution by concentrating sound and focusing the irradiation light, is advancing. In Japanese Translation of PCT Application No. 2011-519281, the resolution is improved by focusing the irradiation light with a lens and disposing the object at the focus position of the light.

The absorption characteristic of the light absorber inside the object differs depending on a wavelength. Accordingly, it is possible to obtain a distribution related to a substance concentration in the object by applying light beams having different wavelengths to the object and performing an operation based on the signal strength of the photoacoustic wave of each wavelength. Specifically, by using an absorption coefficient value of light in the object determined for each wavelength and wavelength dependence of light absorption peculiar to a target substance, the distribution related to the concentration of the substance is visualized. Further, it is possible to acquire an oxygen saturation of blood based on the concentration of oxyhemoglobin HbO and the concentration of deoxyhemoglobin Hb.
Patent Literature 1: Japanese Translation of PCT Application No. 2011-519281

### SUMMARY OF THE INVENTION

When the object is a living body, there are cases where the object moves during photoacoustic measurement due to pulsation, respiration, or other body motions. As a result, a relative positional relationship among the object, the ultrasound probe, and a light irradiation unit is shifted. In addition, in the case of a hand-held photoacoustic apparatus of which the probe is gripped by an operator and in the case of a photoacoustic apparatus that visualizes a wide area by causing the ultrasound probe to move (scan), even when the object does not move, when the ultrasound probe moves from an ideal position, the relative positional relationship mentioned above is shifted. Further, there are cases where the positional relationship among individual portions inside the object is changed due to a deformation of the object caused by the body motion.

When the body motion or the movement of the ultrasound probe described above occurs in a period between measurement with a first wavelength and measurement with a second wavelength during measurement of the oxygen saturation or the like, a shift or a deformation occurs between photoacoustic images derived from the respective wavelengths. Hereinafter, such shift and deformation are collectively referred to as a “position shift”. In the case where the position shift between the wavelengths occurs, the light absorbers at different positions in the object are compared with each other when the substance concentration is calculated. As a result, there is a possibility that accuracy in concentration calculation is reduced.

In particular, in the case where a target light absorber is blood, when the positional relationship is shifted between measurement wavelengths, the position of a vessel is shifted at the respective wavelengths. With this, the ratio of the absorption coefficients between the wavelengths at each vessel position has a wrong value, and hence a wrong oxygen saturation is determined.

Note that the position shift in the present specification means that the position of the object, the position of the ultrasound probe, and the relative positional relationship therebetween are shifted from design values of the apparatus and set values for the scanning. For example, in the case where scanning with the ultrasound probe is performed, detection of the photoacoustic wave is performed at each position on a scanning locus, and a measurement position changes every time the detection is performed. However, such a position change is known information, and hence the position change can be reflected in image reconstruction, and is not referred to as the position shift.

In the conventional photoacoustic apparatus, the operator cannot obtain information on an error in characteristic information such as the oxygen saturation caused by the position shift. When the operator performs interpretation of the obtained photoacoustic image without information related to reliability of a quantitative value of the characteristic information such as the oxygen saturation, there is a possibility that accuracy in diagnosis is reduced.

The present invention has been made in view of the above problem. An object of the present invention is to provide information related to the error in the characteristic information caused by the position shift of the object in the photoacoustic imaging.

The present invention provides an object information acquiring apparatus comprising:

a light source that generates light having a first wavelength and light having a second wavelength;

a conversion element that receives an acoustic wave generated in response to an irradiation of an object with the light having the first wavelength and outputs a first reception signal, and receives an acoustic wave generated in response to an irradiation of the object with the light having the second wavelength and outputs a second reception signal;

a characteristic information acquiring unit that acquires a first characteristic information distribution based on the first reception signal, acquires a second characteristic information distribution based on the second reception signal, and acquires a substance concentration distribution inside the object based on the first and second characteristic information distributions;

a position shift acquiring unit that acquires a position shift between the first characteristic information distribution and the second characteristic information distribution; and

a display controlling unit that outputs an image based on the substance concentration distribution and the position shift, to a displaying unit.

The present invention also provides a control method of an object information acquiring apparatus including a light source, a conversion element, a characteristic information acquiring unit, a position shift acquiring unit, and a display controlling unit, comprising the steps of:

causing the light source to generate light having a first wavelength and light having a second wavelength;

causing the conversion element to receive an acoustic wave generated in response to an irradiation of an object with the light having the first wavelength and to output a first reception signal, and to receive an acoustic wave generated in response to an irradiation of the object with the light having the second wavelength and to output a second reception signal;

causing the characteristic information acquiring unit to acquire a first characteristic information distribution based on the first reception signal, acquire a second characteristic information distribution based on the second reception signal, and acquire a substance concentration distribution inside the object based on the first and second characteristic information distributions;

causing the position shift acquiring unit to acquire a position shift between the first characteristic information distribution and the second characteristic information distribution; and causing the display controlling unit to output an image based on the substance concentration distribution and the position shift, to a displaying unit.

According to the present invention, it is possible to provide information related to the error in the characteristic information caused by the position shift of the object in the photoacoustic imaging.

Further features of the present invention will become apparent from the following description of exemplary embodiments with reference to the attached drawings.

### BRIEF DESCRIPTION OF THE DRAWINGS

FIG. 1 is a schematic view showing an overall configuration of a photoacoustic apparatus of Embodiment 1;

FIG. 2 is a flowchart showing an example of an object information acquiring flow of Embodiment 1;

FIG. 3 is a flowchart showing another example of the object information acquiring flow of Embodiment 1;

FIG. 4 is a flowchart showing an example of an object information acquiring flow of Embodiment 2;

FIG. 5 is a flowchart showing an example of an object information acquiring flow of Embodiment 3;

FIGS. 6A and 6B are schematic views showing examples of a display method of Embodiment 1;

FIGS. 7A and 7B are schematic views showing examples of a display method of Embodiment 2; and

FIG. 8 is a flowchart showing an example of an object information acquiring flow of Embodiment 4.

### DESCRIPTION OF THE EMBODIMENTS

Hereinbelow, preferred embodiments of the present invention will be described with reference to the drawings. Note that the dimensions, materials, shapes, and relative dispositions of components described below should be appropriately changed according to the configuration of an apparatus to which the invention is applied and various conditions. Therefore, the scope of the invention is not limited to the following description. In addition, the same components are designated by the same reference numerals in principle, and the description thereof will be omitted.

The present invention relates to a technique for detecting an acoustic wave that propagates from an object and generating characteristic information of the inside of the object. Therefore, the present invention is viewed as an object information acquiring apparatus or a control method thereof, or an object information acquiring method or a signal processing method. In addition, the present invention is also viewed as a program that causes an information processing apparatus including hardware resources such as a CPU and a memory to execute the methods, or a storage medium that stores the program.

The object information acquiring apparatus of the present invention includes an apparatus that receives the acoustic wave generated by a photoacoustic effect in the object irradiated with light (electromagnetic wave) and acquires object information as image data. Such an apparatus can be also referred to as a photoacoustic apparatus or a photoacoustic imaging apparatus. The characteristic information is information corresponding to each of a plurality of positions in the object that is generated by using a reception signal obtained by receiving a photoacoustic wave.

In the characteristic information acquired by the present invention, an absorption amount and an absorption ratio of light energy are reflected. For example, the characteristic information includes a generation source of the acoustic wave resulting from light irradiation, an initial sound pressure in the object (generated sound pressure), a light energy absorption density and a light energy absorption coefficient derived from the initial sound pressure, and information related to the concentration of a substance constituting a tissue. Examples of the information related to the concentration of the substance include the concentration of oxyhemoglobin or deoxyhemoglobin, a total hemoglobin concentration derived therefrom, and an oxygen saturation. In addition, the information related to the concentration of the substance may include a glucose concentration, a collagen concentration, a melanin concentration, and the volume fraction of fat or water. A two-dimensional or three-dimensional object information distribution is obtained based on the characteristic information at each position in the object. Distribution data can be generated as image data to be displayed in a displaying apparatus.

The acoustic wave in the present invention is typically an ultrasound wave, and includes an elastic wave called a sound wave or an acoustic wave. An electric signal converted from the acoustic wave by a probe or the like is also referred to as an acoustic signal. Note that the description of the ultrasound wave or the acoustic wave in the present specification is not intended to limit the wavelength of the elastic wave. The acoustic wave generated by the photoacoustic effect is referred to as a photoacoustic wave or an optical ultrasound wave. An electric signal derived from the photoacoustic wave is also referred to as a photoacoustic signal.

The object information acquiring apparatus of the present invention is capable of measuring a living body of a human or an animal, samples other than the living body, and a calibration sample such as a phantom. In the case where the object is the living body, the object information acquiring apparatus is expected to be used in the diagnosis of a vascular disease and a malignant tumor.

### Embodiment 1

Hereinbelow, the configuration and processing of an object information acquiring apparatus (photoacoustic apparatus) of Embodiment 1 will be described.

(Apparatus Configuration)

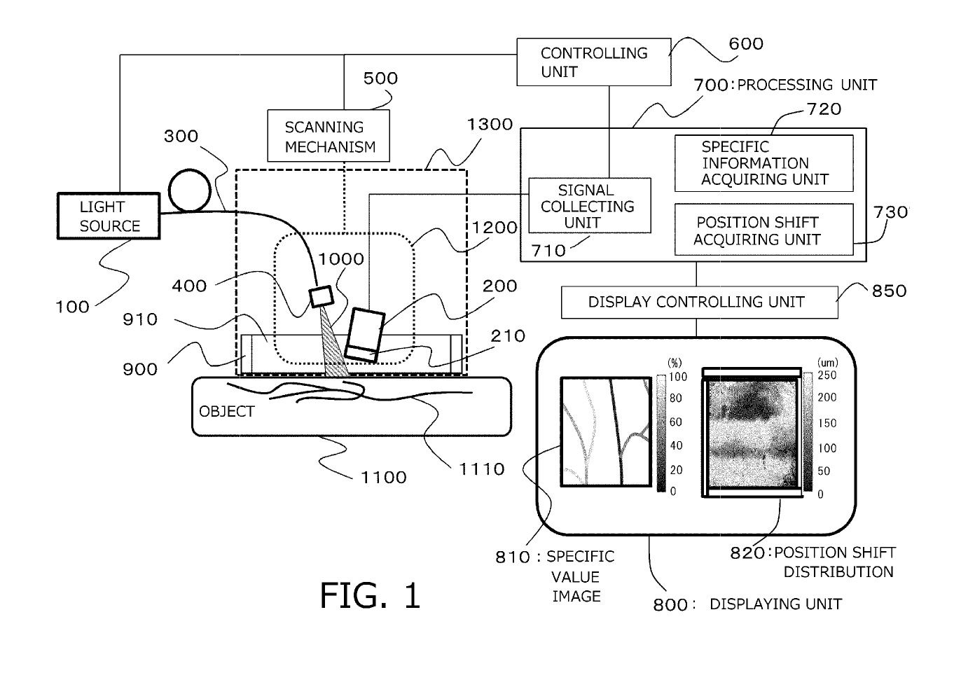

FIG. 1 is a schematic view showing the configuration of the photoacoustic apparatus of the present embodiment. As the basic components, the apparatus includes a light source 100, a probe 200, a light guiding unit 300, a light irradiation unit 400, a scanning mechanism 500, a controlling unit 600, a processing unit 700, a displaying unit 800, and a water tank 900. The processing unit 700 includes a signal collecting unit 710, a characteristic information acquiring unit 720, and a position shift acquiring unit 730. The probe 200 includes a conversion element 210.

Pulsed light emitted from the light source 100 is applied to an object 1100 as irradiation light 1000 from the light irradiation unit 400 through the light guiding unit 300, and reaches a light absorber 1110 in the object 1100. Examples of the light absorber 1110 are typically hemoglobin, a vessel that contains a large amount of hemoglobin, and a tumor involving neovascularization in a living body. The light absorber 1110 absorbs light energy and generates a photoacoustic wave. The generated photoacoustic wave propagates in the object and reaches the conversion element 210.

The conversion element 210 outputs a time-series reception signal by receiving the photoacoustic wave. To the processing unit 700, the reception signal outputted from the conversion element 210 is sequentially inputted. Note that, in the present embodiment, the conversion element 210 (reception surface) of the probe 200 is soaked in water 910 as an acoustic matching member in the water tank 900. With this, acoustic matching of the object 1100 and the conversion element 210 is achieved.

The scanning mechanism 500 moves a measuring unit 1200 that includes the probe 200 and the light irradiation unit 400 to change the relative positional relationship with respect to the object 1100. The controlling unit 600 controls individual configuration blocks in the photoacoustic apparatus.

The processing unit 700 generates characteristic information related to the absorption rate of light by using the signal inputted from the conversion element 210. In addition, the processing unit 700 calculates the characteristic information related to the concentration such as the oxygen saturation and a position shift of the object between measurement wavelengths based on the characteristic information related to the absorption rate of light obtained for each measurement wavelength. The processing unit 700 transmits data on the generated characteristic information and the position shift to a display controlling unit 850. The display controlling unit 850 causes a displaying unit 800 to display an image of the characteristic information and information related to the position shift.

Hereinbelow, the detail of each configuration block of the photoacoustic apparatus according to the present embodiment will be described.

(Light Source 100)

As the light source 100, a pulsed light source capable of generating pulsed light on the order of nanoseconds to microseconds is preferable. As the specific pulse width, the pulse width of about 1 to 100 nanoseconds is preferable. In addition, as the wavelength, the wavelength of about 400 nm to 1600 nm is preferable. In particular, when the vessel in the vicinity of the surface of the living body is imaged with high resolution, the wavelength in a visible light region (not less than 400 nm and not more than 700 nm) is preferable. On the other hand, when the deep portion of the living body is imaged, the wavelength with which an absorption amount is small in a background tissue of the living body (not less than 700 nm and not more than 1100 nm) is preferable. Note that it is also possible to use regions of a terahertz wave, a microwave, and a radio wave.

As the specific light source 100, a laser is preferable. In order to output light beams having a plurality of wavelengths that include at least a first wavelength and a second wavelength, a variable wavelength laser capable of changing an oscillation wavelength is more preferable. As the laser, it is possible to use a solid laser, a gas laser, a dye laser, and a semiconductor laser. In particular, a pulsed laser such as a Nd:YAG laser or an alexandrite laser is preferable. In addition, a Ti:sa laser, an optical parametric oscillators (OPO) laser, or the dye laser that uses Nd:YAG laser light as excitation light may also be used. A plurality of light sources having different wavelengths may be used. In addition, it is also possible to use a light-emitting diode or a flash lamp instead of the laser.

(Probe 200)

The probe 200 includes at least one conversion element 210 and a casing that supports the conversion element 210. As the conversion element 210, it is possible to use an element that receives the acoustic wave and converts the acoustic wave to an electric signal. Examples of the conversion element 210 include a piezoelectric element that uses piezoelectricity of lead zirconate titanate (PZT), a conversion element that uses light resonance, and a capacitive conversion element such as a CMUT.

In the case where the photoacoustic apparatus is a photoacoustic tomography apparatus, it is preferable to provide a plurality of the conversion elements 210 in the probe 200. The plurality of the conversion elements 210 are preferably disposed so as to be arranged in a plane called 1D array, 1.5D array, 1.75D array, or 2D array or an arc-shaped or bowl-shaped curved plane. On the other hand, in the case where the photoacoustic apparatus is a photoacoustic microscope, the probe 200 is preferably a focus-type probe. In that case, an acoustic lens is provided on the reception surface of the conversion element 210.

In addition, in order to stabilize the shape of the object, it is preferable to provide a holding member that is not shown. In the case of the bowl-shaped probe, a dish-shaped or cup-shaped holding member is preferable. Further, it is also possible to use a configuration in which the object is held between two plate-like members. As the material of the holding member, a material that transmits light and the acoustic wave is preferable. When the holding member is used, an advantage that the calculation of a light amount distribution described later can be simplified is achieved.

The scanning mechanism 500 mechanically moves the probe 200 with respect to the object 1100, whereby it is possible to acquire the object information of a wade area. The light irradiation unit 400 and the probe 200 preferably move in synchronization with each other. As a scanning method, in accordance with the shape of the probe or the object, it is possible to use a raster scan, a snake scan, and a spiral scan. In the case where the probe 200 is a hand-held probe, the probe 200 has a grip which the operator uses to grip the probe 200.

(Light Guiding Unit 300)

The light guiding unit 300 transmits light from the light source 100 to the light irradiation unit 400. As the light guiding unit 300, it is possible to use optical elements such as an optical fiber, a lens, a mirror, a prism, and a diffusion plate.

(Light Irradiation Unit 400)

The light irradiation unit 400 applies light transmitted by the light guiding unit 300 to the object 1100 as the irradiation light 1000. Herein, in the photoacoustic tomography apparatus, the light irradiation unit 400 preferably increases the diameter of the beam using a lens or the like and performs the irradiation. On the other hand, in the photoacoustic microscope, in order to increase the resolution, the light irradiation unit of the light guiding unit 300 is preferably constituted by a lens or the like, and the irradiation light 1000 is preferably focused and applied.

In addition, the light irradiation unit 400 may be moved with respect to the object 1100. Further, the light irradiation unit 400 may also be moved in synchronization with the probe 200. With this, it is possible to visualize a wider area. In the case of the bowl-shaped probe, the light irradiation unit 400 may be disposed at the center of the bowl. Note that it is also possible to apply light to the object 1100 directly from the light source 100 without using the light guiding unit 300 and the light irradiation unit 400.

(Processing Unit 700)

The processing unit 700 of the present embodiment includes the signal collecting unit 710, the characteristic information acquiring unit 720, and the position shift acquiring unit 730.

The signal collecting unit 710 collects a time-series analog reception signal (referred to as a photoacoustic signal) outputted from the conversion element 210. The signal collecting unit 710 performs signal processing such as amplification of the reception signal, AD conversion of the analog reception signal, and storage of a digitized reception signal. In addition, the signal collecting unit 710 collects a signal related to a light amount (referred to as a light amount signal) outputted from a photodetector (not shown) into which part of the irradiation light 1000 enters for each irradiation pulse. As the signal collecting unit 710, it is possible to use a circuit called a data acquisition system (DAS). The signal collecting unit 710 is constituted by, e.g., an amplifier that amplifies the reception signal, an AD converter, and the like. Note that the amplifier may be provided in the probe 200. In the present invention, light having the first wavelength is converted to a first reception signal, and light having the second wavelength is converted to a second reception signal.

The characteristic information acquiring unit 720 acquires the characteristic information related to the absorption rate of light in the object for each position by using the photoacoustic signal and the light amount signal collected by the signal collecting unit 710. For example, a generated sound pressure distribution, a light energy absorption density distribution, and a light absorption coefficient distribution are obtained.

Further, the characteristic information acquiring unit 720 determines the characteristic information related to the concentration of the substance present in the object (especially an oxygen saturation distribution of blood) by using the characteristic information related to the absorption rate of light of each wavelength. As an image reconstruction method at the time of acquisition of the characteristic information, it is possible to use known methods such as universal back projection (UBP), filtered back projection (FBP), and delay and sum. In the present invention, a first characteristic information distribution is acquired from the first reception signal, and a second characteristic information distribution is acquired from the second reception signal. In addition, a substance concentration distribution is acquired from the first characteristic information distribution and the second characteristic information distribution. There are cases where the distribution of the position shift is used in the acquisition of the substance concentration distribution.

The position shift acquiring unit 730 calculates the position shift between the measurement wavelengths by using the characteristic information related to the absorption rate of light of each measurement wavelength calculated in the characteristic information acquiring unit 720.

As each of the characteristic information acquiring unit 720 and the position shift acquiring unit 730, it is possible to use a processor such as a CPU or a GPU, or an arithmetic circuit such as a field programmable gate array (FPGA) chip. Note that each of the characteristic information acquiring unit 720 and the position shift acquiring unit 730 may be constituted by one processor or one arithmetic circuit, and may also be constituted by a plurality of processors or a plurality of arithmetic circuits. In addition, each of the characteristic information acquiring unit 720 and the position shift acquiring unit 730 may include a memory that stores the reception signal, generated distribution data and display image data, and various measurement parameters. The memory is typically constituted by at least one storage medium such as a ROM, a RAM, or a hard disk.

(Displaying Unit 800)

The displaying unit 800 displays the characteristic information related to the concentration of the substance such as the oxygen saturation calculated in the characteristic information acquiring unit 720 and the position shift distribution between the measurement wavelengths calculated in the position shift acquiring unit 730. As the displaying unit 800, it is possible to use a liquid crystal display (LCD), a cathode ray tube (CRT), and an organic EL display. The displaying unit 800 may be provided separately from the photoacoustic apparatus. As the display controlling unit 850, it is possible to use an information processing apparatus and a control circuit. Alternatively, a control circuit that is disposed in the displaying unit may also be used.

(Water Tank 900)

The water tank 900 is a container capable of holding the water 910 as the acoustic matching member. By soaking the conversion element 210 provided in the probe 200 in the water 910, it is possible to acoustically match the object 1100 and the conversion element 210. The surface of the water tank 900 that comes in contact with the object 1100 is preferably formed of a film thinner than the wavelength of the photoacoustic wave such that the photoacoustic wave passes through the surface easily. In addition, the acoustic matching member and a contact surface are preferably formed of a material that does not easily absorb the irradiation light 1000. As the acoustic matching member, water, ultrasound gel, or oil is suitable. In addition, as the material of the contact surface, it is possible to use polyethylene, urethane rubber, and PET. The acoustic matching member such as the ultrasound gel or water may be appropriately provided between the object 1100 and the contact surface.

(Scanning Mechanism 500)

As the scanning mechanism 500, it is possible to use an automatic stage that includes a stepping motor or a servomotor. In addition, the scanning mechanism 500 can be constituted by combining mechanical components such as an XY stage, a shaft, and a screw mechanism, and a position detection mechanism and a position control mechanism of the probe or the light irradiation unit. FIG. 1 shows the configuration in which the scanning mechanism 500 moves the measuring unit 1200 to scan measurement points on the object 1100. However, as long as the measurement can be performed while the measurement points on the object 1100 are scanned, any configuration may be adopted. For example, a configuration in which the irradiation light 1000 is applied to a wide area of the object 1100, and the scanning mechanism 500 moves only the probe 200 may be adopted.

Conversely, a configuration in which the probe capable of receiving the photoacoustic wave in a wide area (e.g., a single transducer or an array transducer having a wide focus area) is used as the probe 200, and the scanning mechanism 500 moves only the light irradiation unit may also be adopted. In this case, it is preferable to cause the irradiation light 1000 to condense. Note that, in the case where the probe 200 is fixed, the acoustic matching member does not need to be a liquid. For example, instead of the water tank 900 and the water 910, it is possible to use a gel member (polyurethane-based gel or the like).

In addition, the scanning mechanism 500 can perform the scanning with the angle of the probe 210 or the light irradiation unit 400 being changed. Further, the scanning mechanism 500 can scan the measurement points on the object 1100 without directly moving the probe 200 or the light irradiation unit 400. For example, by controlling a mirror that reflects the photoacoustic wave and the irradiation light 1000 (changing the angle or moving the mirror), it is possible to scan the irradiation position of the irradiation light 1000 and the detection position of the photoacoustic wave. In this case as well, the light irradiation position, the detection position of the photoacoustic wave, or both of them may serve as the movement target. As such a mirror, a galvanometer mirror and an MEMS mirror are suitable.

(Controlling Unit 600)

The controlling unit 600 supplies necessary control signals and data to the individual configuration blocks. Specifically, the controlling unit 600 supplies the signal to instruct the light source 100 to emit light, the reception control signal of the conversion element 200, and the control signal of the scanning mechanism 500. Further, the controlling unit 600 performs signal amplification control, AD conversion timing control, and storage control of the reception signal. The controlling unit 600 can also be constituted by one processor such as the CPU or the GPU or one circuit such as the FPGA chip, or by combining a plurality of the processors or a plurality of the circuits, similarly to the processing unit 700. In addition, the controlling unit 600 may include a memory that stores various measurement parameters. The memory is typically constituted by at least one storage medium such as the ROM, the RAM, or the hard disk. The memory can be shared with the processing unit 700.

(Object 1100)

Although the object 1100 does not constitute part of the photoacoustic apparatus of the present invention, the object 1100 will be described below. The main purpose of the photoacoustic apparatus according to the present embodiment is the diagnosis of a vascular disease and a malignant tumor of a human or an animal or a follow-up of chemotherapy. Therefore, the object 1100 includes the living body, specifically, diagnosis target portions such as a breast, a neck, an abdomen, a face, and a skin of a human body or an animal. In the living body, a body motion occurs due to respiration or pulsation, a shift from the disposition position or a deformation tends to occur. Note that the position shift or the deformation can occur even in the case of a non-living object. Note that there are cases where the scanning mechanism moves the object, but this movement does not correspond to the position shift of the present invention. The position shift in the present invention denotes a state in which the target of the object is moved from an estimated position in the case where image reconstruction is performed from the photoacoustic signals derived from a plurality of wavelengths.

As the light absorber 1110 inside the object 1100, the one having a relatively high light absorption coefficient inside the object 1100 is preferable. For example, when the human body is the measurement target, oxyhemoglobin, deoxyhemoglobin, a vessel containing a large amount of oxyhemoglobin or deoxyhemoglobin, or a malignant tumor including many neovascular vessels serves as the target of the light absorber 1110. In addition, a melanoma or a plaque in a carotid wall serves as the target of the light absorber 1110.

(Hand-Held Type)

The present invention can also be applied to the hand-held photoacoustic apparatus. In this case, a member surrounded by a dotted line 1300 may be appropriately stored in one casing that can be gripped by the operator.

(Object Information Acquiring Method)

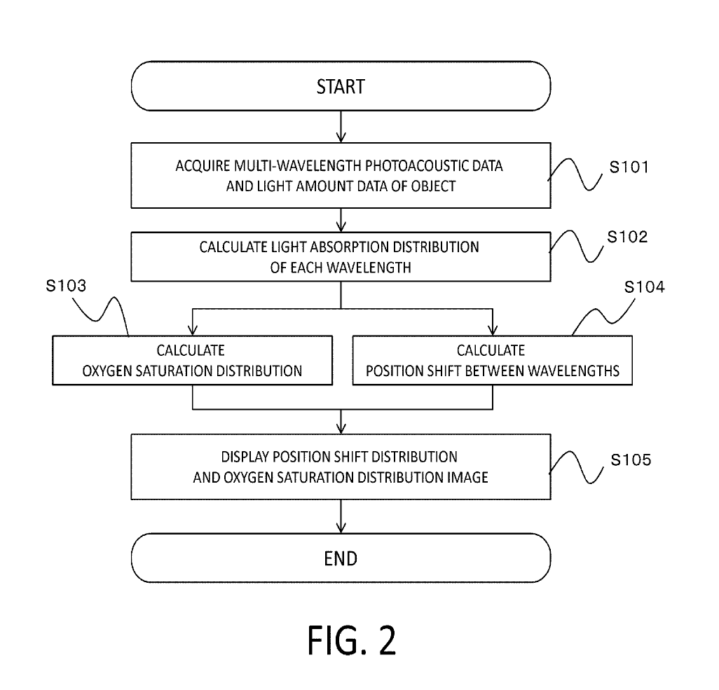

An example of the flow in which the processing unit 700 collects measurement data, calculates the characteristic information of the object and the position shift, and displays them in the displaying unit 800 in the photoacoustic apparatus according to the present embodiment will be described by using FIG. 2. Herein, the oxygen saturation is taken as an example of the characteristic information related to the concentration, but other characteristic information can also be acquired by the same flow. The present flow is started at the point of time when the object is disposed in a measurable area and preparation processes such as activation and idling of the apparatus are completed.

In Step S101, multi-wavelength photoacoustic data and light amount data are acquired. First, the light irradiation unit 400 applies light to the object. The conversion element 210 receives the photoacoustic wave generated at each light pulse. The signal collecting unit 710 collects the time-series analog reception signal outputted from the conversion element 210 for each channel, performs various signal processing on the time-series analog reception signal, and retains the signal. In addition, the signal collecting unit 710 collects the light amount signal of each pulse outputted from the photodetector. At this point, in order to obtain the image in the wire area, the probe 200 and the light irradiation unit 400 are moved relative to the object by the scanning mechanism 500, and the photoacoustic waves are received at a plurality of scanning positions.

By performing the above processes for a plurality of wavelengths, Step S101 is completed. Note that, in the present flow, the measurement with the second wavelength is performed after the photoacoustic data and the light amount data with the first wavelength are acquired. However, during the movement of the probe 200 by the scanning mechanism 500, the measurement with the first wavelength and the measurement with the second wavelength may be performed alternately. In this case as well, since the body motion can occur during the measurement with a plurality of the wavelengths, the present invention exerts the effect. In addition, with regard to the light amount, an estimated light amount value pre-stored in a memory may be acquired instead of actually measuring the light amount. The estimated light amount value can be calculated based on the configuration of the apparatus, a light source control value, the positional relationship between the object and the light irradiation unit, and the depth of a target voxel in the object.

In Step S102, the characteristic information acquiring unit 720 calculates the light absorption distribution data in the object for each measurement wavelength by performing analysis by using the photoacoustic signal and the light amount signal obtained in S101. That is, the characteristic information acquiring unit 720 acquires the light absorption distribution data at each measurement position by correcting the generated sound pressure generated from the photoacoustic signal by the image reconstruction based on the light amount signal.

An example of a correction method will be described. First, the characteristic information acquiring unit 720 calculates the light amount of the irradiation light 1000 applied to the object 1100 from the light amount signal. At this point, the irradiation light amount is calculated by using the relationship between the light amount signal and the irradiation light amount that are measured in advance. It is preferable to have the relationship between the light amount signal and the irradiation light amount as a relation table or a relation expression of the light amount signal and the irradiation light amount. Subsequently, the characteristic information acquiring unit 720 calculates the light amount distribution data in the object based on the irradiation light amount and the shape of the object. At this point, it is possible to perform calculation that uses a finite element method or a Monte Carlo method based on a light transport equation or a light diffusion equation. By measuring the irradiation distribution of the irradiation light in advance, it is possible to acquire more accurate light amount distribution data.

Thereafter, by dividing the generated sound pressure distribution data by the light amount distribution data, the light absorption distribution data in the object or data proportional to the light absorption distribution data in the object is acquired. Note that, in the case where the measurement is performed while the scanning is performed in the photoacoustic tomography apparatus, the generated sound pressure distribution data and the light amount distribution data at each scanning position are calculated by using the photoacoustic signal and the light amount signal outputted from the signal collecting unit 710 at each scanning position. Thereafter, based on each scanning position information, by dividing total generated sound pressure distribution data obtained by totaling the generated sound pressure distribution data at the individual scanning positions by total light amount distribution data obtained by totaling the light amount distribution data at the individual scanning positions, it is possible to acquire the light absorption distribution data in the object.

On the other hand, in the case where the photoacoustic apparatus is the photoacoustic microscope, the characteristic information acquiring unit 720 performs envelop detection on the photoacoustic signal outputted from the signal collecting unit 710 with respect to a temporal change. Subsequently, the characteristic information acquiring unit 720 converts a time axis direction in the signal of each light pulse to a depth direction, and performs plotting on spatial coordinates. By performing this process for each measurement position (scanning position), the sound pressure distribution data is acquired.

Further, the characteristic information acquiring unit 720 corrects the sound pressure distribution data at each measurement position by using the light amount signal outputted from the signal collecting unit 710 to acquire the light absorption distribution data in the object. For example, in the case where the photodetector is a photodiode, the peak value of the reception signal outputted from the photodiode at each measurement point is acquired. Subsequently, by dividing the sound pressure data by the peak value, the light absorption distribution data in the object or data proportional thereto is acquired.

In Step S103, the characteristic information acquiring unit 720 determines the oxygen saturation distribution (substance concentration distribution) of blood by using the light absorption distribution data of each wavelength determined in S102.

On the other hand, in Step S104, the position shift acquiring unit 730 calculates the position shift between the measurement wavelengths by using the light absorption distribution data of each measurement wavelength determined in S102. Hereinbelow, an example of a position shift calculation method will be described. One of the light absorption distribution data sets of two wavelengths determined in S102 is used as a reference image. The light absorption distribution of the other wavelength (referred to as a deformed image) is subjected to deformable registration so as to match the reference image. Specifically, a correlation between the images is calculated at a point extracted at random in volume data of the light absorption distribution. Subsequently, the image is deformed and optimized such that the correlation is increased. At this point, it is possible to use an index indicative of the degree of a match between the reference image and the deformed image such as normalized cross-correlation.

For the deformation, it is possible to use known methods such as free form deformation. Note that the deformable registration may be performed stepwise. For example, after registration is roughly performed by performing rotation, scaling, shearing, and parallel moving by affine conversion, the free form deformation is used. Note that, in the present embodiment, the light absorption distribution is used in the correction of the position shift without altering the light absorption distribution, but an unnecessary portion may be removed, and processing in which the light absorption distribution is converted to a logarithmic image of the light absorption distribution may be performed in advance.

In the deformed image having been subjected to the deformable registration in this manner, the amount of movement of each point in the volume data from the position before the deformable registration to the position after the deformable registration is assumed to be the position shift. As the position shift, it is possible to use a linear movement distance of each point from the position before the deformation to the position after the deformation, a movement amount in a specific direction (e.g., a vertical direction relative to the reception surface of the conversion element 210), and a vector quantity having direction components. Note that, in the measurement with three or more wavelengths as well, it is possible to perform the same deformable registration by using the light absorption distribution of a given measurement wavelength as the reference image.

In Step S105, the oxygen saturation distribution calculated in S103 and the position shift distribution between the measurement wavelengths calculated in S104 are displayed in the displaying unit 800. As a display method of both images, a superimposition display, an alternate display, and a display with notes that use letters and signs may be used in addition to a display in which the images are arranged side by side as shown in FIG. 1. In addition, the display method may be changed in accordance with the instruction of the operator that uses an inputting apparatus such as a mouse or a keyboard.

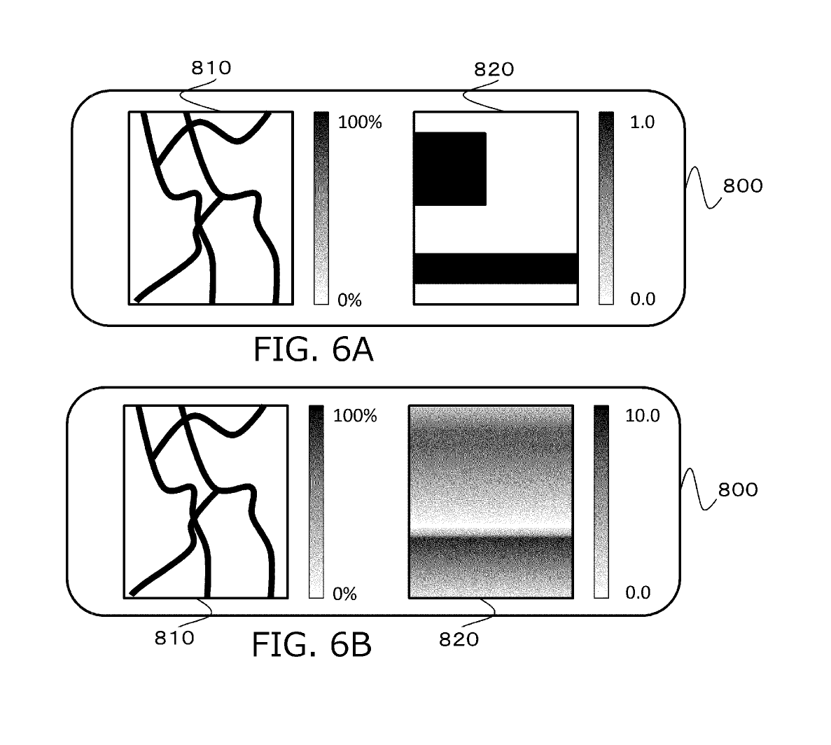

An example of the display method of the position shift distribution includes a method that directly displays the position shift, as shown in FIG. 1. In the direct display method, the position shift is preferably associated with lightness, chroma, or hue. All of these methods may be used in combination. In addition, as shown in FIG. 6A, a given threshold value of the position shift may be provided, and a position shift (820) that is binarized according to whether or not the position shift is not less or not more than the threshold value may be displayed together with the characteristic information (810). Further, as shown in FIG. 6B, an error (820) in the characteristic information related to the concentration caused by the position shift may be calculated based on the determined position shift, and may be displayed together with the characteristic information (810).

In the case of the photoacoustic microscope, it is preferable to display the position shift distribution in a depth direction (the vertical direction relative to the reception surface of the conversion element 210). This is because the shift of the focus position of the ultrasound wave caused by the position shift in the depth direction significantly changes the received sound pressure and, by extension, increases the error in the characteristic information.

After the processes up to Step S105 by the photoacoustic apparatus are ended, the operator performs image interpretation based on the characteristic information related to the concentration of the substance such as the oxygen saturation and the position shift distribution between the measurement wavelengths that are displayed in S105. At this point, an oxygen saturation distribution image may be appropriately interpreted with the position shift used as the index of reliability.

<Modification>

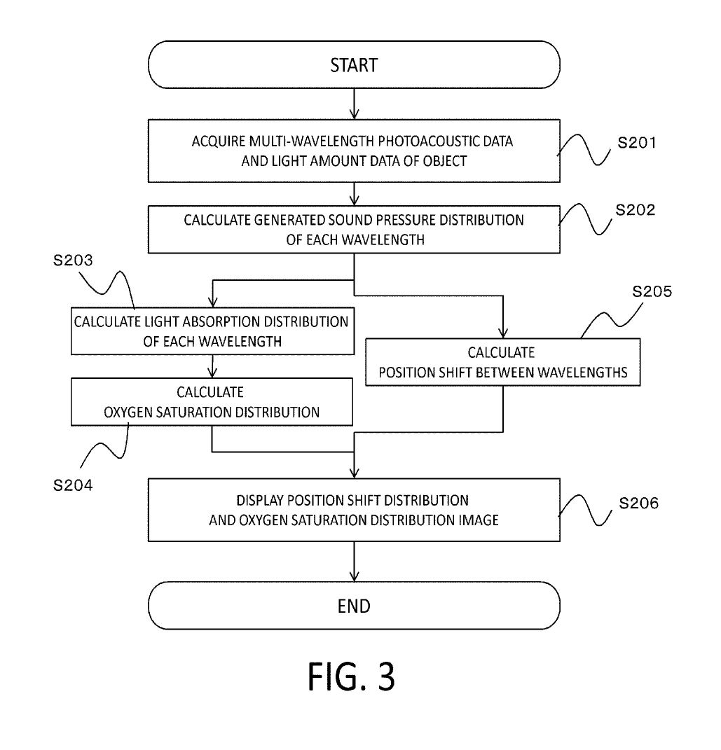

In the present embodiment, the position shift between the measurement wavelengths is calculated by using the light absorption distribution data of each measurement wavelength in S104. However, another characteristic information may also be used. For example, the flow when the position shift acquiring unit 730 calculates the position shift by using the generated sound pressure distribution data is shown in FIG. 3.

In S202, the characteristic information acquiring unit 720 calculates the generated sound pressure distribution data. In S205, the position shift acquiring unit 730 calculates the position shift by using the generated sound pressure distribution data. The calculation method of the position shift is the same as that when the light absorption distribution data is used. On the other hand, in 5203 to 5204, the oxygen saturation distribution (substance concentration distribution) is determined based on the light absorption distributions derived from light beams having a plurality of wavelengths. In the other Steps, the same processes as those in FIG. 2 are performed.

Instead of using the characteristic information related to the light absorption ratio, for example, the object 1100 may be imaged by a camera or the like at the time of the measurement at each wavelength, and the position shift of the object between the measurement wavelengths may be calculated based on camera images.

Thus, according to the present embodiment, it is possible to provide the information related to the error in the characteristic information caused by the position shift of the object. In particular, it is possible to display the information related to the reliability of the characteristic information resulting from the position shift in comparison with the reconstructed image, and hence the present embodiment is useful for the image interpretation of the operator.

### Embodiment 2

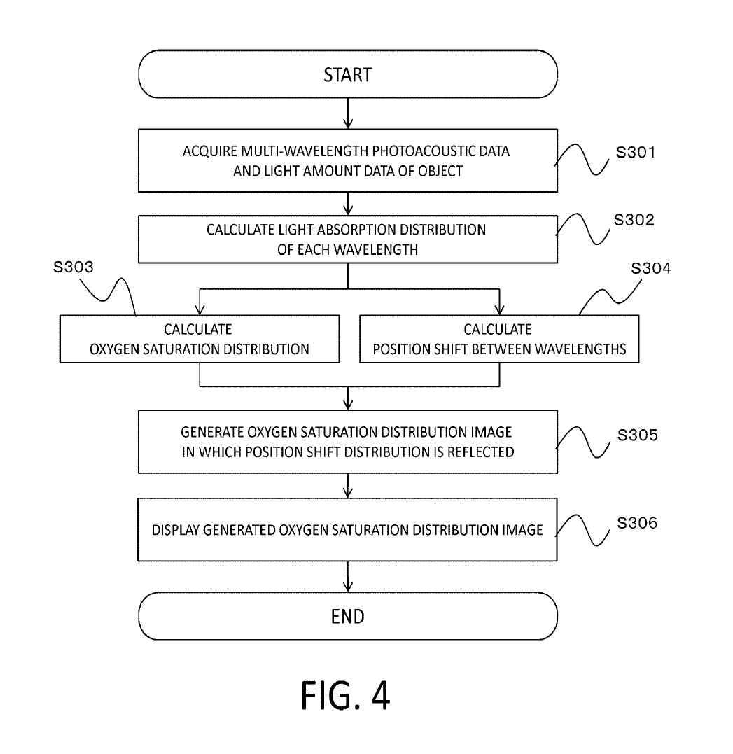

The configuration of the photoacoustic apparatus of the present embodiment is the same as that in Embodiment 1. Hereinbelow, the processing detail of the present embodiment will be described with reference to the flow in FIG. 4 with a focus on a part of the processing detail different from that in Embodiment 1. Processes in Steps S301 to S304 are the same as those in Steps S101 to S104 in Embodiment 1.

In Step S305, the characteristic information acquiring unit 720 generates characteristic information related to the concentration of the substance in which the position shift distribution is reflected by using the oxygen saturation distribution obtained in S303 and the position shift distribution between the measurement wavelengths obtained in S304.

For example, a given threshold value of the position shift is provided, and the characteristic information related to the concentration of the substance is assumed to be 0 or null (no information) with regard to a portion in which the position shift is not less than the threshold value. With this, in the next S306, with regard to a portion in which the position shift is large and the error in the characteristic information is large, the characteristic information related to the concentration of the substance is not displayed. Consequently, only the characteristic information at the position in which the error is small and the reliability is high is displayed. It is preferable to set the threshold value of the position shift in accordance with the resolution of the photoacoustic apparatus. For example, in the case of the photoacoustic microscope having a resolution of 50 μm, it is preferable to set the threshold value to 50 μm.

As another example, there is a method in which the characteristic information related to the concentration of the substance is weighted in accordance with the position shift distribution. For example, the characteristic information related to the concentration of the substance is assumed to be hue data, and the position shift is allocated as lightness data. At this point, the lightness data is generated such that lightness is increased as the position shift becomes smaller, and the lightness is reduced as the position shift becomes larger. With this, in the next S306, a portion in which the position shift is small, i.e., a portion in which the error in the value is small and the reliability is high is emphasized.

In the case of the photoacoustic microscope, it is preferable to generate the characteristic information related to the concentration of the substance in which the position shift distribution is reflected by using the position shift distribution in the depth direction (the vertical direction relative to the reception surface of the conversion element 210).

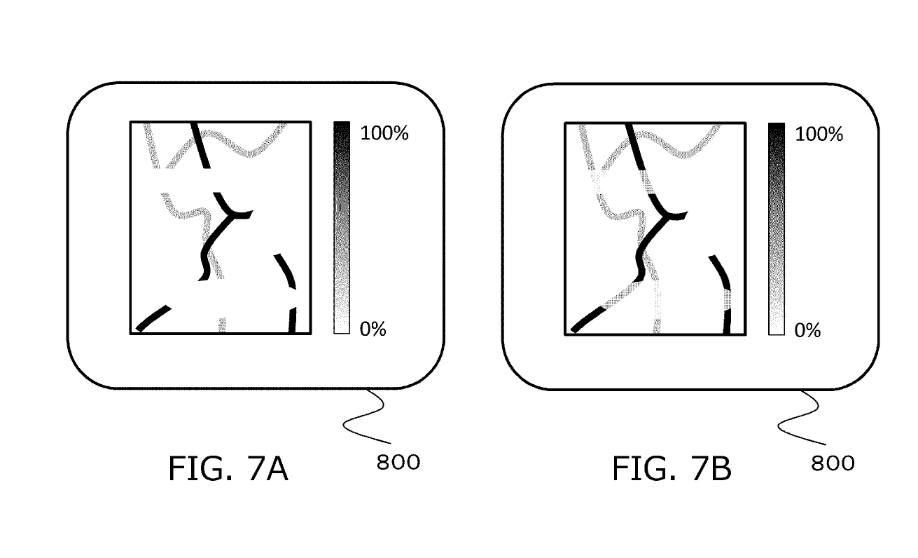

In Step S306, the characteristic information generated in S305 in which the position shift distribution is reflected is displayed in the displaying unit 800. For example, in FIG. 7A, the characteristic information of only a portion in which the position shift is not more than a predetermined threshold value is displayed. In FIG. 7B, the lightness is adjusted in accordance with the position shift, and the characteristic information data related to the concentration is displayed as the hue data.

Thus, according to the present embodiment, the information related to the error in the quantitative value of the characteristic information such as the oxygen saturation caused by the position shift is given to the operator. In particular, in the present embodiment, the index of the reliability of the image is displayed so as to be superimposed on the reconstructed image of the inside of the object, and it is possible to understand the reliability of each portion at sight.

### Embodiment 3

### Calculate Oxygen Saturation after Position Shift Correction

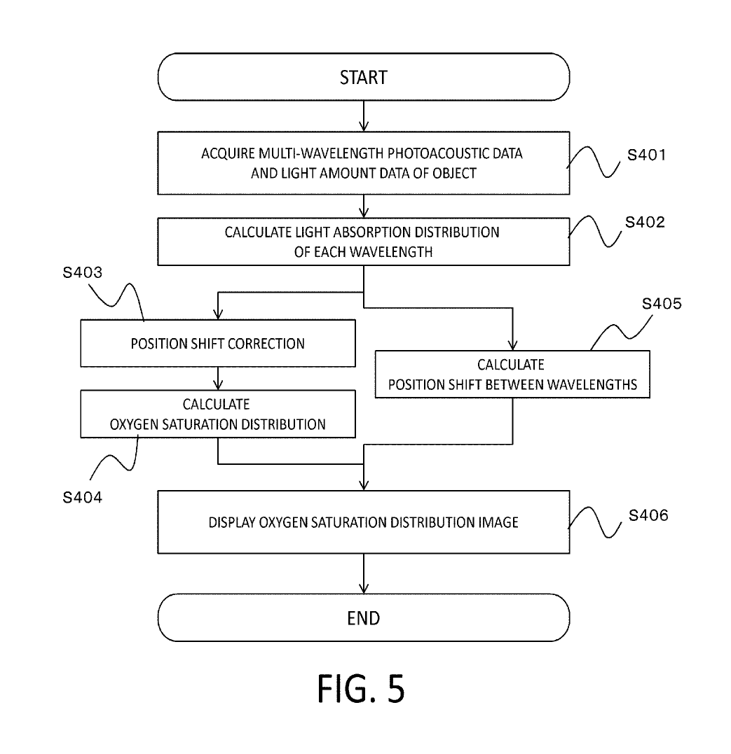

The configuration of the photoacoustic apparatus of the present embodiment is the same as that in each embodiment described above. Hereinbelow, the processing detail of the present embodiment will be described with reference to the flow in FIG. 5 with a focus on a part of the processing detail different from that in each embodiment described above. Processes in Steps S401 to S402 are the same as those in Steps S101 to S102 in Embodiment 1.

In Step S403, the characteristic information acquiring unit 720 performs the deformation and the registration to the position shift of the light absorption distribution data of each measurement wavelength determined in S402. Hereinafter, the deformation and the registration between the measurement wavelengths is referred to as a position shift correction. In the position shift correction, when the position shift is detected, it is possible to use the method of the deformable registration described in Embodiment 1. The characteristic information acquiring unit 720 modifies the positions of pixels of the image that is not the reference image based on the detected position shift.

In Step S404, the characteristic information acquiring unit 720 determines the substance concentration distribution such as the oxygen saturation distribution of blood by using the light absorption distribution data of each wavelength after the position shift correction performed in S403.

In Step S405, the position shift acquiring unit 730 calculates the position shift between the measurement wavelengths by using the light absorption distribution data of each wavelength determined in S402. The calculation method of the position shift is the same as that in Step S104 in Embodiment 1. In addition, in the present embodiment, the position shift correction between the measurement wavelengths is performed in S403, and hence the position shift distribution may be determined by comparing data before the position shift correction with data after the position shift correction.

In Step S406, the substance concentration distribution such as the oxygen saturation distribution is displayed in the displaying unit 800. Note that, in the present embodiment, since the position shift is already corrected, only the oxygen saturation distribution may be displayed in order to simplify the display. However, the position shift distribution may also be displayed for the purpose of reference. As the display method of both images, any method such as the side-by-side display, the superimposition display, or the alternate display described above may be used. The display method may be switched by the operation of a user. In addition, in the alternate display, the image may be switched and displayed automatically, or the display image may also be switched by the operation of the user. Further, as in Embodiment 2, the position shift and the oxygen saturation (the characteristic information value) may be reflected in the hue and the like and displayed.

After the processes up to Step S406 by the photoacoustic apparatus are ended, the operator interprets the oxygen saturation distribution image. At this point, since the position shift is already corrected, the operator can acquire the object inside information having high accuracy only by glancing at the displayed image. In the case where the position shift distribution is displayed together with the oxygen saturation distribution, the accuracy of the diagnosis is further improved by using the position shift distribution as the index of the reliability. Note that, in the present embodiment as well, various characteristic information values and the camera image may be used for the calculation and correction of the position shift.

In the present embodiment, since the characteristic information related to the concentration is calculated after the position shift correction is performed, the error in the value caused by the position shift is small. This effect is conspicuous in the case of the photoacoustic tomography. On the other hand, in the case of the photoacoustic microscope, the ultrasound focus position shift caused by the position shift in the depth direction (a directivity axis direction of an element) is reflected in the received sound pressure and the characteristic information value sensitively. In addition, in the case where the characteristic information related to the concentration is calculated after the registration is performed, the position shift can be corrected but the absolute value of the sound pressure cannot be corrected, and hence the error in the value occurs in accordance with the position shift. For the above-described reasons, the present embodiment is particularly suitable for the photoacoustic microscope.

Thus, according to the photoacoustic apparatus according to the present embodiment, it is possible to provide the information related to the error in the quantitative value of the characteristic information such as the oxygen saturation caused by the position shift. In particular, since it is possible to present the image in which the position shift is already corrected, the operator can easily view and intuitively understand the image.

### Embodiment 4

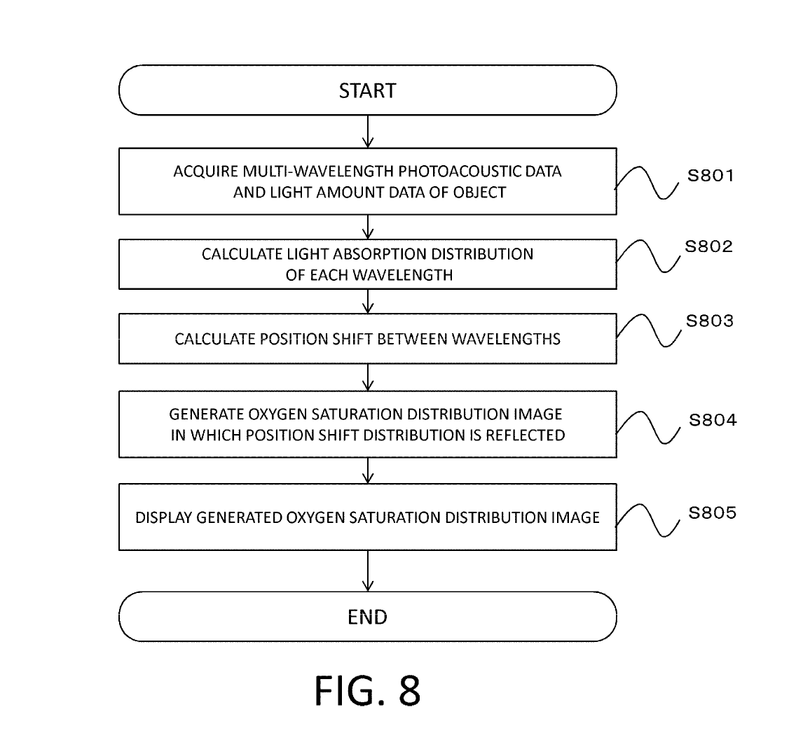

Hereinbelow, an example of the processing flow of Embodiment 4 will be described by using FIG. 8. In the present embodiment, as the light source 100, a Ti:sa laser is used. Laser light is applied to the surface of the object 1100 by using an optical fiber as the light guiding unit 300. As the probe 200, a probe in which 512 piezo elements as the conversion elements 210 are disposed spirally on a semi-spherical supporter is used. The semi-spherical supporter is scanned on an XY plane by a scanner (the scanning mechanism 500) that moves along an X axis and a Y axis.

The signal collecting unit 710 of the present embodiment has a function of simultaneously receiving all data from acoustic wave detection elements of 512 channels, performing the amplification and digital conversion of the data, and transferring the data to a computer as the characteristic information acquiring unit 720. The sampling frequency of the signal collecting unit 710 is 20 MHz, and uses the timing of the light irradiation as a reception start timing.

The object 1100 is a phantom made of a semi-spherical urethane rubber that simulates a living body. In the phantom, titanium oxide as a scatterer and two types of inks that simulate an absorption spectrum of blood as the absorber are mixed. In addition, a spherical block rubber having a diameter of 0.5 mm is buried as the light absorber 1110 at the center in the phantom. The size of the phantom is 80 mm in diameter. The phantom is fixed with a transparent plastic cup (holding member), and is in contact with the probe 200 via water as the acoustic matching member.

In Step S801, while scanning by the semi-spherical probe is performed along a circumferential or spiral track, pulsed light beams having wavelengths of 756 nm and 797 nm are alternately applied to the phantom at 10 Hz by using two Ti:sa lasers. Subsequently, the probe 200 acquires the photoacoustic signal at each measurement position. In addition, similarly to Embodiment 1, the light amount data at each measurement position is acquired. Note that, depending on the accuracy of the scanning mechanism, an error of about ±200 μm can occur with respect to a specified measurement position. Next, in Step S802, the characteristic information acquiring unit 720 calculates the light absorption distribution at each measurement position for each wavelength by a method similar to that in Embodiment 1.

In Step S803, the position shift acquiring unit 730 calculates the position shift between the wavelengths. Herein, in the light absorption distribution calculated for each wavelength at each measurement position, the light absorption distribution measured with the wavelength of 797 nm is set as the reference image. Subsequently, the movement amount that maximizes the correlation value of the reference image and the light absorption distribution data with the wavelength of 756 nm is defined as the shift. With this, the shift (or shift vector) in each of X, Y, and Z directions at each measurement position is calculated.

Note that this description corresponds to the case of a step and repeat method in which the scanning mechanism 500 repeats the stop and movement of the probe and the photoacoustic measurement with each wavelength is performed at each stop position. However, the present embodiment can also be applied to the case where the photoacoustic measurement is performed while the probe continuously moves by performing proper interpolation calculation.

In S804, the characteristic information acquiring unit 720 shifts the light absorption distribution data with the wavelength of 756 nm in the X, Y, and Z directions based on the shift calculated in S803 to correct the shift. Subsequently, the characteristic information acquiring unit 720 calculates the oxygen saturation distribution image from the reference image and the light absorption distribution data with the wavelength of 756 nm of which the shift correction is completed. With this, the oxygen saturation distribution image in which the position shift distribution is reflected is generated.

In Step S805, the generated oxygen saturation distribution image is displayed in the displaying unit 800. The display method may be the same as that in each embodiment described above. With this, when the image interpretation is performed by the operator, the oxygen saturation distribution image having high accuracy in which the shift is corrected is displayed. Note that the position shift distribution (vector) between the measurement wavelengths calculated in the position shift acquiring unit 730 may be displayed together with the oxygen saturation as the information related to the reliability.

Thus, according to the present invention, it becomes possible to give the information related to the error in the quantitative value of the characteristic information such as the oxygen saturation caused by the position shift of the object to the operator. Since the operator can perform the image interpretation by using the above information as the index of the reliability of the image, more accurate diagnosis is allowed.

Thus, the present invention has been described in detail with reference to the specific embodiments. However, the present invention is not limited to the specific embodiments described above, and the embodiments can be modified within the scope of the technical idea of the present invention.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-Ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-198218, filed on Oct. 6, 2015, which is hereby incorporated by reference herein in its entirety.

What is claimed is:

1. An object information acquiring apparatus comprising:
a light source that generates light having a first wavelength and light having a second wavelength;
a conversion element that receives an acoustic wave generated in response to an irradiation of an object with the light having the first wavelength and outputs a first reception signal, and receives an acoustic wave generated in response to an irradiation of the object with the light having the second wavelength and outputs a second reception signal;
a characteristic information acquiring unit that acquires a first characteristic information distribution based on the first reception signal, acquires a second characteristic information distribution based on the second reception signal, and acquires a substance concentration distribution inside the object based on the first and second characteristic information distributions;
a position shift acquiring unit that acquires a position shift between the first characteristic information distribution and the second characteristic information distribution; and
a display controlling unit that outputs an image based on the substance concentration distribution and the position shift, to a displaying unit.

2. The object information acquiring apparatus according to claim 1, wherein
the position shift acquiring unit acquires the position shift based on a change in a relative position between the object and the conversion element in a period between irradiation with the light having the first wavelength and irradiation with the light having the second wavelength.

3. The object information acquiring apparatus according to claim 1, wherein
the position shift acquiring unit acquires the position shift caused by a body motion of the object in a period between irradiation with the light having the first wavelength and irradiation with the light having the second wavelength.

4. The object information acquiring apparatus according to claim 1, wherein
the position shift acquiring unit acquires the position shift caused by a deformation of the object in a period between irradiation with the light having the first wavelength and irradiation with the light having the second wavelength.

5. The object information acquiring apparatus according to claim 1, wherein
the display controlling unit causes the displaying unit to display an image representing the substance concentration distribution and an image representing a distribution of the position shift side by side.

6. The object information acquiring apparatus according to claim 1, wherein
the display controlling unit causes the displaying unit to display an image representing the substance concentration distribution and an image representing a distribution of the position shift in a superimposed manner.

7. The object information acquiring apparatus according to claim 5, wherein
the display controlling unit causes the displaying unit to display an image in which the distribution of the position shift is associated with at least any of lightness, chroma, and hue.

8. The object information acquiring apparatus according to claim 5, wherein
the position shift acquiring unit acquires the distribution of the position shift that is binarized.

9. The object information acquiring apparatus according to claim 5, wherein
the position shift acquiring unit acquires an error in the substance concentration distribution caused by the position shift based on the distribution of the position shift.

10. The object information acquiring apparatus according to claim 1, wherein
the characteristic information acquiring unit acquires the substance concentration distribution that is corrected based on the position shift.

11. The object information acquiring apparatus according to claim 10, wherein
the characteristic information acquiring unit deforms the second characteristic information distribution based on the position shift such that the second characteristic information distribution matches the first characteristic information distribution, and acquires the substance concentration distribution by using the deformed second characteristic information distribution and the first characteristic information distribution.

12. The object information acquiring apparatus according to claim 11, wherein
the display controlling unit causes the displaying unit to display only a portion of the substance concentration distribution in which the position shift is not more than a predetermined threshold value.

13. The object information acquiring apparatus according to claim 11, wherein
the display controlling unit causes the displaying unit to display the substance concentration distribution that is weighted based on a distribution of the position shift.

14. The object information acquiring apparatus according to claim 1, wherein
the position shift acquiring unit acquires the position shift based on a correlation between the first characteristic information distribution and the second characteristic information distribution.

15. The object information acquiring apparatus according to claim 1, wherein
the position shift acquiring unit acquires the position shift based on a camera image.

16. The object information acquiring apparatus according to claim 1, wherein
the characteristic information acquiring unit acquires an oxygen saturation distribution as the substance concentration distribution.

17. A control method of an object information acquiring apparatus including a light source, a conversion element, a characteristic information acquiring unit, a position shift acquiring unit, and a display controlling unit, comprising the steps of:
causing the light source to generate light having a first wavelength and light having a second wavelength;
causing the conversion element to receive an acoustic wave generated in response to an irradiation of an object with the light having the first wavelength and to output a first reception signal, and to receive an acoustic wave generated in response to an irradiation of the object with the light having the second wavelength and to output a second reception signal;
causing the characteristic information acquiring unit to acquire a first characteristic information distribution based on the first reception signal, acquire a second characteristic information distribution based on the second reception signal, and acquire a substance concentration distribution inside the object based on the first and second characteristic information distributions;
causing the position shift acquiring unit to acquire a position shift between the first characteristic information distribution and the second characteristic information distribution; and
causing the display controlling unit to output an image based on the substance concentration distribution and the position shift, to a displaying unit.
